(19) United States
(12) Patent Application Publication
(10) Pub. No.: US 2004/0241766 A1
(43) Pub. Date: Dec. 2, 2004

(54) Carbamazepine assay

(76) Inventors: Sara Rathman Zwart, Houston, TX (US); Robert McMahon, Evansville, IN (US)

Correspondence Address: Amy A. Ostrom, Ph.D., Akerman Senterfitt, Suite 400, 222 Lakeview Avenue, West Palm Beach, FL, 33401-6183, US

(21) Appl. No.: 10/737,112

(22) Filed: Dec. 16, 2003

Related U.S. Application Data
(60) Provisional application No. 60/474,091, filed on May 28, 2003.

(51) Int. Cl. G01N 33/53; C12P 19/60

(52) U.S. Cl. 435/7.5

(57) ABSTRACT

Methods and assays for detecting ureido group-containing compounds and quantifying levels of these compounds in a sample are described herein.

### CROSS REFERENCE TO RELATED APPLICATION

This application claims priority from U.S. provisional patent application Ser. No. 60/474,091, filed May 28, 2003.

### FIELD OF THE INVENTION

The invention relates generally to the fields of medicine and chemistry. More particularly, the invention relates to methods and devices for detecting and quantifying ureido group-containing compounds in a sample.

### BACKGROUND

Carbamazepine (CBZ), also known as 5-carbamoyl-5H-dibenz[b,f]azepine; carbazepine, 5H-Dibenz[b,f]azepine-5-carboxamide, and Tegretol® (Novartis, Basel, Switzerland), is an anticonvulsant drug that has been used for decades to manage epilepsy and other disorders such as trigeminal neuralgia and mood disorders. To determine patient compliance and proper dosing, physicians collect and send a blood sample to an analytical lab where high-performance liquid chromatography (HPLC) or gas chromatography (GC) is used to determine serum CBZ concentrations. These conventional methods are expensive, time-consuming, and not amenable to performance in a typical physician's office.

### SUMMARY

The invention relates to the discovery that CBZ, a ureido group-containing compound, binds directly to avidin. Based on this discovery, methods and assays for detecting and quantifying CBZ were developed that are simpler and less expensive to perform than conventional HPLC or GC methods. Moreover, the simplicity of the methods and assays of the invention allow CBZ to be measured on site at a physician's office or a patient's home.

Accordingly, the invention features a method for detecting CBZ in a sample that includes the steps of providing a test sample, adding to the test sample an agent including avidin, and measuring the amount of CBZ specifically bound to avidin. The test sample can be serum, for example. The avidin can be avidin from an egg, streptavidin, and avidin derivatives, and can be labeled with a detectable label. A variation of the foregoing method includes the additional steps of providing at least one immobilized agent that binds avidin, and contacting the test sample and the agent including avidin to the at least one immobilized agent that binds avidin under conditions that allow binding of avidin to CBZ and binding of avidin to the at least one immobilized agent. In this method, the steps of providing at least one immobilized agent that binds avidin and contacting the test sample and the agent including avidin to the at least one immobilized agent that binds avidin under conditions that allow binding of avidin to CBZ and binding of avidin to the at least one immobilized agent are performed prior to the step of measuring the amount of CBZ specifically bound to avidin. In preferred variations of the invention, the step of measuring the amount of CBZ specifically bound to avidin includes measuring the amount of avidin bound to the at least one immobilized agent. The at least one immobilized agent can be biotin, which can be conjugated to bovine serum albumin (BSA). The combination of biotin and bovine serum albumin can be contacted with a plastic dish. The avidin can be labeled with a detectable label such as a radioisotope, a fluorescent compound, a bioluminescent compound, a chemiluminescent compound, colloidal gold, a magnetic particle, and an enzyme. Such detectable label can be horseradish peroxidase.

In a variation of the foregoing methods, the step of contacting the test sample and the agent including avidin to the at least one immobilized agent that binds avidin under conditions that allow binding of avidin to CBZ and binding of avidin to the at least one immobilized agent includes mixing the test sample and the avidin before contacting the test sample and the avidin to the at least one immobilized agent that binds avidin. Measuring the amount of avidin bound to the at least one immobilized agent can include contacting the avidin bound to the at least one immobilized agent with a substrate molecule that interacts with the detectable label yielding a detectable signal. In one example of this method, the level of signal is proportional to the level of CBZ in the test sample. Such detectable signal can be light, color or radioactivity. The detectable signal can be quantified using a spectrophotometer.

In preferred variations of the invention, the steps of providing a test sample, adding to the test sample an agent including avidin, and measuring the amount of CBZ specifically bound to avidin are performed in a competitive binding assay. Such competitive binding assay can be a radioimmunoassay, radioligand binding assay, fluorescence polarization binding assay, ELISA, microplate reader-based assay, fluorimetric displacement assay, FRET assay, affinity chromatography-based assay, non-chromatographic affinity assay, protein microarray assay, or fluorimmunoassay.

In other variations of the invention, the steps of providing a test sample, adding to the test sample an agent including avidin, and measuring the amount of CBZ specifically bound to avidin are performed in a non-competitive binding assay. Such non-competitive binding assay can be an agglutination assay, enzyme immunoassay, immunometric assay, radio immunoassay, ELISA, fluorescent immunoassay, chemiluminescent assay, calorimetric assay, plasmon resonance assay, FRET assay, lateral flow assay, or flow cytometry assay.

In another aspect, the invention includes a kit for detecting CBZ in a sample. The kit includes a plurality of immobilized agents that bind avidin, a plurality of label-conjugated avidins, a wash solution, a reagent for detection of the complexes formed between the label-conjugated avidins and the plurality of immobilized agents that bind avidin, and instructions for use. The plurality of immobilized agents that bind avidin can be conjugated to a solid support (e.g., a plastic dish).

The present invention provides assays and methods for detecting CBZ in a sample. For example in an embodiment of the invention, a sample containing CBZ and free biotin is mixed with avidin in a first vessel. A portion of the avidin molecules bind to all or a portion of the CBZ molecules. The avidin is bound to a detectable label. The contents of the first vessel are added to a second vessel containing an immobilized agent that specifically binds avidin. The immobilized agent displaces CBZ that is bound to avidin. Avidin molecules that are not bound to immobilized agents are then removed. Avidin molecules that are specifically bound to immobilized agents are measured by quantifying the amount of signal emitted by the detectable label. The greater the amount of CBZ present in the sample, the greater the amount of avidin that binds the immobilized agent. The amount of avidin bound to the immobilized agent is proportional to the level of signal emitted by the detectable label. The number of avidin molecules that specifically bind to the immobilized agents are quantified, resulting in the quantification of CBZ in the sample.

Unless otherwise defined, all technical terms used herein have the same meanings as commonly understood by one of ordinary skill in the art to which this invention belongs. Commonly understood definitions of medical terms can be found in Thomas Lathrop Stedman, Stedman's Medical Dictionary, Lippincott, Williams & Wilkins: Philadelphia, Pa., 2000. Commonly understood definitions of chemistry terms can be found in Richard J. Lewis, Hawley's Condensed Chemical Dictionary, 14th ed., John Wiley & Sons: Hoboken, N.J., 2001.

The terms “bind,” “binds,” or “interacts with” means that one molecule recognizes and adheres to a particular second molecule in a sample, but does not substantially recognize or adhere to other structurally unrelated molecules in the sample. Generally, a first molecule that “specifically binds” a second molecule has a binding affinity greater than about 10⁵ to 10⁶ liters/mole for that second molecule.

The term “immobilized” refers to an agent that is coupled, either chemically or non-chemically, to a support. Examples of supports include, but are not limited to, plates (e.g., plastic or glass plates), membranes, beads and particles.

Although methods and materials similar or equivalent to those described herein can be used in the practice or testing of the present invention, suitable methods and materials are described below. All publications, patent applications, patents and other references mentioned herein are incorporated by reference in their entirety. In the case of conflict, the present specification, including definitions will control. The particular embodiments discussed below are illustrative only and not intended to be limiting.

### BRIEF DESCRIPTION OF THE DRAWINGS

FIG. 1 is a graph of a standard curve developed with known concentrations of CBZ.

FIG. 2 is a diagram of steps in a method of measuring CBZ levels.

### DETAILED DESCRIPTION

The invention provides methods and assays for detecting ureido group-containing compounds (e.g., CBZ) and quantifying levels of these compounds in a sample (e.g., urine, serum) based on the discovered ability of CBZ to directly bind avidin. The below described preferred embodiments illustrate adaptations of these methods and compositions. Nonetheless, from the description of these embodiments, other aspects of the invention can be made and/or practiced based on the description provided below.

### Avidin, Avidin Substitutes and Avidin Derivatives

The assays and methods for assaying CBZ are based on the discovery that CBZ binds directly to avidin. As with biotin, the ureido group of CBZ is believed to mediate its interaction with avidin. Accordingly, any agent that selectively binds CBZ by its ureido group might be used in the invention. Given their well-defined use in biotin-based assays, particularly preferred agents that selectively bind CBZ are avidin (i.e., avidin from an egg), avidin substitutes, and avidin derivatives. Avidin substitutes or derivatives include, for example, streptavidin, avidin derivatives that feature low non-specific binding properties and avidin derivatives having a reduced affinity for biotin molecules above a pH of 9.

### Detecting CBZ in a Sample

An exemplary method of the invention for detecting CBZ in a sample includes the steps of providing a test sample, adding to the test sample an agent including avidin, and measuring the amount of CBZ specifically bound to avidin. The test sample can be any substance which is suspected of containing CBZ or known to contain CBZ. The test sample can be untreated (undiluted), or chemically and/or physically treated, diluted, or concentrated prior to analysis. Examples of samples include, but are not limited to, samples from biological sources such as physiological fluids, including blood, plasma, serum and any other type of fluid, tissue or material which is suspected of containing CBZ.

Assays of the invention for detecting CBZ in a sample typically involve a solid support on which is immobilized an agent, e.g., biotin, that specifically binds either avidin or CBZ. As one example, avidin and a test sample suspected of containing CBZ are added to a support containing immobilized biotin. The immobilized biotin competes for binding sites on avidin and displaces CBZ bound to avidin. Thus, more avidin molecules bind the immobilized biotin when the sample contains greater amounts of CBZ. In another example, CBZ is immobilized on a solid support. The test sample is added to the support along with labeled soluble avidin. The immobilized CBZ competes with any CBZ in the test sample for binding the labeled avidin. Thus, where greater amounts of CBZ are present in the test sample, lower levels of labeled avidin bound to the immobilized CBZ are observed.

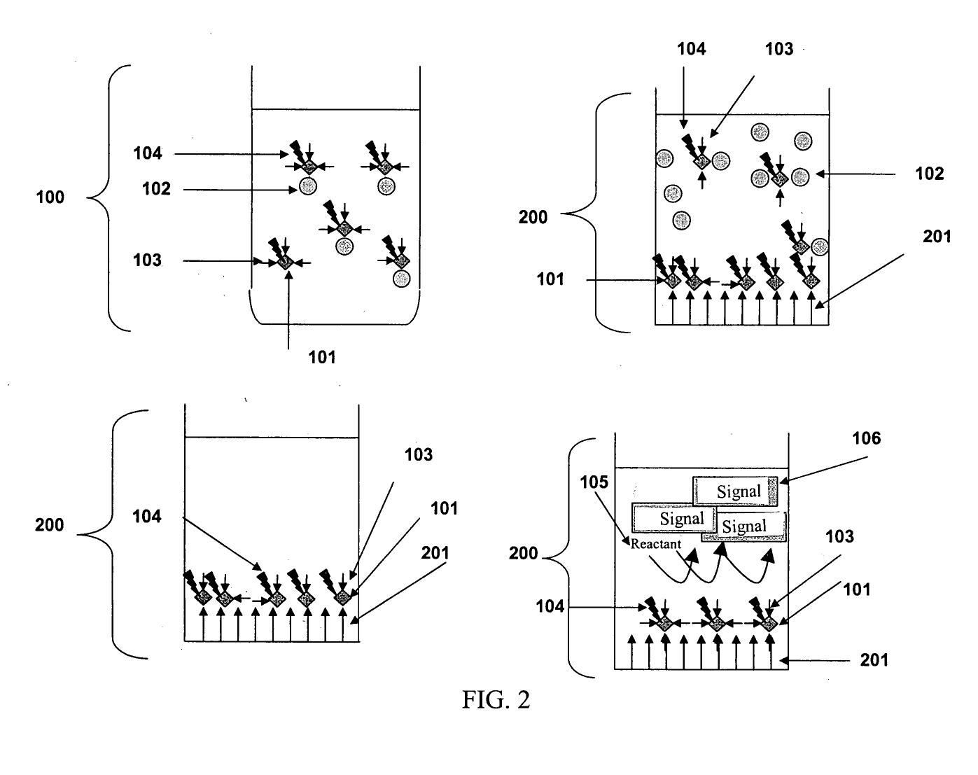

Competitive binding assays are preferred in the invention. One example of a competitive binding assay for detecting CBZ in a sample is shown in FIG. 2. The initial step in a method of detecting CBZ involves mixing avidin 101 with a sample containing CBZ 102 and free biotin 103 in vessel 100. In the vessel, a portion of the avidin molecules bind to all or a portion of the CBZ molecules. A sufficient amount of avidin 101 is added to bind all biotin 103 in the sample. To measure the amount of avidin in the sample, the avidin 101 can be bound to a detectable label 104. A second step in the assay involves adding the contents of vessel 100 to second vessel 200. Second vessel 200 contains immobilized agent 201 that specifically binds to avidin 101. Once these reagents are brought together, appropriate reactions occur. Specifically, since immobilized agent 201 competes for binding sites on avidin 101, immobilized agent 201 displaces CBZ 102 bound to avidin 100. Hence, the more CBZ 102 present in the sample, the more avidin 101 binds the immobilized agent 201. Following competition of the immobilized agent 201 for available binding sites on avidin 101, the avidin 101 that is not bound to immobilized agent 201 is removed (e.g., by washing). The quantity of avidin 101 molecules that are specifically bound to the immobilized agent 201 are measured by detecting and quantifying the amount of signal emitted by the detectable label 104 in vessel 200. Detection of label 104 may be performed by any suitable technique, e.g., spectrophotometry, luminometry, densitometry (e.g., optical densitometry), etc.

In typical embodiments of the above method, the reagents are brought together at a suitable temperature, generally in the range of from about 0° C. to about 45° C., and preferably at 25° C. The time for mixing can vary from a few seconds to 24 hours, although typically the mixing step requires at least 60 minutes.

The immobilized agent 201 can be any agent capable of binding to avidin or CBZ. A notable example of such an agent is biotin. In a preferred embodiment, the immobilized agent 201 is BSA-conjugated biotin (i.e., biotinylated BSA). However, other agents that may be used include biotin conjugated to other non-biotin proteins, biotinylated nucleotides, biotinylated antibodies (e.g., Goldman et al., Phys. Stat. Sol. 229:407414, 2002; Renukaradhya et al., Rev. Sci. Tech. 20:749-756, 2001; BD Biosciences (San Jose, Calif.); Pierce Biotechnology (Rockford, Ill.); Prozyme (San Leandro, Calif.)), and biotin derivatives. A non-exhaustive list of biotin derivatives includes biotin dimers and trimers, Co-reduced N-aminohexyl biotinamido (Sabatino et al., J. Med. Chem. 46:3170-3173, 2003), biotinyl p-nitrophenyl ester (Pazy et al., J. Biol. Chem. 277:30892-308900, 2002), homobiotin (Wilbur et al., Bioconjug. Chem. 12:616-623, 2001), deferoxamine acetyl-cysteinylbiotin (Hashmi and Rosebrough, Drug Metab. Dispos. 23:1362-1367, 1995), biotin derivatives having a carbodiimide function (Masuda et al., Nucleic Acids Symp. Ser. 34:69-70, 1995), +-biotinyl-3-maleimidopropionamidyl-3,-6-dioxaoctane diamine (Spura et al., J. Biol. Chem. 275:22452-22460, 2000), biocytin (Wada et al., J. Biochem. (Tokyo) 123:946-952, 1998), biotin-BMCC (Kamiya, T., J. Immunoassay 18:111-123, 1997) and biotin chelates (Virzi et al., Int. J. Rad. Appl. Instrum: B. 18:719-726, 1991). A number of reagents for biotinylating proteins and antibodies are commercially available (e.g., EZ-Link® Sulfo-NHS-LC-Biotin by Pierce Biotechnology, Rockford, Ill.).

A detectable label 104 of the invention can be any label that allows detection of the substance to which the label is bound. Examples of suitable detectable labels include a radioisotope, a fluorescent compound, a bioluminescent compound, a chemiluminescent compound, colloidal gold, a magnetic particle, a photocleavable moiety and/or an enzyme. Label 104 can be present on avidin 101 or the immobilized agent 201. In one embodiment, the detectable label is a reporter enzyme. Reporter enzymes of the invention include, but are not limited to, peroxidases, glucose oxidases, β-galactosidases, adenosine deaminases, ureases, alkaline phosphatases, creatine kinase, uricase, glucose-6-phosphate dehydrogenases and other reporter enzymes known in the art. A preferred method of the invention uses a horseradish peroxidase as a reporter enzyme. The type of signal generated by the reporter enzyme would depend on the specific reporter enzyme as well as the reagents and substrates used. Signals generated by reporter enzymes can include chemiluminescent, electrochemical, radiologic or colorometric signals.

Chemiluminescent signals can be generated in a wide variety of ways in response to a reporter enzyme. In most chemiluminescent systems, the reporter enzyme is a peroxidase, and an oxidant such as hydrogen peroxide is present or generated in some fashion (for example, the reaction of an oxidase with its substrate). Useful chemiluminescent signals are generated using, for example, acridinium salts, dioxetanes, tetrakis (dimethylamino) ethylene, luciferin, lucigenin, oxalyl chloride, certain oxidases (for example, xanthine oxidase) and 2,3,-dihydro-1,4-phthalazinediones (such as luminol and isoluminol). Many examples of such compounds and their uses are known in the art, for example, in U.S. Pat. No. 4,383,031, U.S. Pat. No. 4,598,044, U.S. Pat. No. 4,729,950, U.S. Pat. No. 5,180,893, and Chemiluminescence in Organic Chemistry (Gundermann et al., Springer-Verlag, Berlin, 1987, pages 204-207). For generating a chemiluminescent signal, the reporter enzyme is preferably a peroxidase and a compound such as luminol is used as the signal-generating reagent.

In a preferred embodiment, a colorimetric signal is generated. Such signals can be achieved using a wide variety of reporter enzymes and reagents, as is well known in the art. Where the reporter enzyme is a peroxidase (e.g., horseradish peroxidase), as is preferred, useful dye-providing reagents include, but are not limited to, o-phenylenediamine, tetramethylbenzidine and derivatives thereof, and triarylmethanes, imidazole leuco dyes, such as the triarylimidazole leuco dyes described in U.S. Pat. No. 4,087,747, and U.S. Pat. No. 5,024,935. Substrate solutions for the various reporter enzymes are provided in a wash solution or at the end of the assay. One useful substrate solution for o-phenylenediamine includes hydrogen peroxide, citric acid, and sodium phosphate. Intensity of colorometric and chemiluminescent signals can be measured using instruments apparent to one of skill in the art such as spectrophotometry. Radioactive labels can be measured using an instrument such as a scintillation counter.

The avidin and reporter enzyme conjugates used in the practice of the invention can be prepared using any conventional technique of the art for covalently binding proteins, hormones, drugs or other chemical or biological compounds. Useful methods of binding include, but are not limited to, binding of peptides, periodate oxidation, use of glutaraldehyde, dication ethers, carbamoylonium salts, carbodiimides or N-hydroxysuccinimide, and others readily apparent to one skilled in the art.

Vessel 100 can be any container suitable for mixing or immobilizing the sample containing CBZ 102 with avidin 101. Second vessel 200 can be any suitable container for immobilizing the immobilized agent 201. The mixing of avidin 101 with the CBZ sample and also the immobilization of the immobilized agent 201 can be carried out in microtiter plates, test tubes, microfuge tubes, and other devices known in the art. In a preferred embodiment, vessel 100 is a U-bottom 96-well microtiter plate and second vessel 200 is a flat bottom 96-well microtiter plate.

Additionally, vessels 100 and 200 can be used in combination with water-insoluble supports (e.g., on which agents are immobilized) such as chromatography beads, microspheres, films, papers, or membranes. If solid supports are utilized for the assay, the supports can be easily separated from the aqueous phase using filtration or other methods known in the art. Supports such as biotin-coated polystyrene plates (e.g., Reacti-Bind™ Biotin Coated Polystyrene Strip Plates, Pierce Biotechnology, Rockford, Ill.) streptavidin agarose, CaptAvidin, and Captivate™ ferrofluid magnetic particles are available from commercial sources (e.g., MoBiTec, Goettingen, Germany).

### Detecting Additional Ureido Group-Containing Compounds in a Sample

In addition to detecting CBZ, methods of the invention can also be used to detect other ureido group-containing compounds in a sample. Examples of other ureido group-containing compounds include primidone, phenytoin, progabide, oxcarbamazepine and phenobarbital.

### Competitive Binding Assays

A myriad of competitive binding assays exist, and any suitable such assay may be used to detect CBZ in a sample. For reviews of such assays, see White H. B., Methods Enzymol. 279:464-466, 1997; Bylund and Trews, Am. J. Physiol. 265:L421-429, 1993; and Schrijver and Kramps, Rev. Sci. Tech. 17:550-561, 1998. Examples of additional competitive binding assays include: chemiluminescence immunoassays involving a competitive reaction between a highly specific monoclonal antibody, free antigen, and solid phase-bound antigen (Neupert et al., Prostaglandins 52:385-401, 1996), microtiter plate binding assays involving immobilization of biotin-BSA on a microtiter plate which takes up avidin-labeled peroxidase due to an avidin-biotin reaction (Chen et al., Anal. Chem. 64:301-323, 1992), fluorimetric displacement assays for biotin-avidin interactions employing 2,6-ANS, competitive enzyme-linked immunosorbent assays (ELISAs) of biotin (Shiuan et al., Methods Enzymol. 279:321-326, 1997; Shrijven and Kramps, Rev. Sci. Tech. 17:550-561, 1998), bioluminescence competitive binding assays for biotin based on photoprotein aequorin (Lizano et al., Methods Enyzmol. 279:296-303, 1997), assays involving avidin or streptavidin-coated beads, biotin/avidin-mediated homogenous assays (Cho et al., Analytical Sciences vol. 15 1999), and assays involving the addition of anti-avidin antibody to a mixture containing CBZ and avidin.

### Non-Competitive Binding Assays

The invention also includes methods of detecting CBZ in a sample involving non-competitive binding assays. Any suitable non-competitive binding assay may be used to detect CBZ in a sample. Many non-competitive binding assays are known in the art and are described in U.S. Pat. No. 5,705,338 and U.S. patent application Ser. No. 10/012,280. Examples of non-competitive binding assays include, but are not limited to: non-competitive ELISAs (Schrijver and Kramps, Rev. Sci. Tech. 17:550-561, 1998), agglutination assays, radioligand binding assays, assays involving molecularly imprinted polymers (Andersson L. I., J. Chromatogr. B Biomed. Sci. Appl. 739:163-173, 2000), homogenous fluorescence polarization binding assays (Lee and Bevis, J. Biomol. Screen 5:415-419, 2000), binding assays involving in vitro selected oligonucleotides as receptors (Ito et al., Methods 22:107-114, 2000), receptor binding assays, enzyme immunoassays (reviewed in Ekins R., Nucl. Med. Biol. 21:495-521, 1994), two-site immunometric assays such as direct immunoassays, radioimmunoassays, fluorescent immunoassays (Goldman et al., Phys. Stat. Sol. vol. 1:407414, 2002), assays involving liposomes coated with fluorescent dyes and a target-specific biotinylated detection reagent, protein microarrays (Templin et al., Drug Discov. Today 7:815-822, 2002), chromatographic and non-chromatographic affinity assays (Labrou and Clonis, J. Biotechnol. 36:95-119, 1994), HPLC/avidin binding assays (Zempleni and Mock, J. Nutr. 129:494S-497S, 1999) chemiluminescent assays and colorimetric assays, binding assays such as plasmon resonance, energy transfer assays, lateral flow assays and flow cytometry assays.

### Kits For Detecting CBZ in a Sample

Also within the invention are kits for detecting and quantifying CBZ levels in a sample. Suck kits contain a conjugate of avidin and a detectable label (e.g., reporter enzyme), and an immobilized agent that specifically binds avidin. In most embodiments, the kits include all of these reagents as well as suitable reagents for providing a colorimetric, fluorometric, chemiluminescent, or other such signal in response to the detectable label (e.g., reporter enzyme), wash solutions, disposable assay devices (e.g., microtiter plates) and instructions for carrying out the method of the invention. Kits may be particularly useful in a hospital or clinical setting by providing an assay that can be performed on-site, resulting in quicker detection of CBZ levels in a sample than can be obtained by sending the sample off-site for analysis.

The below described preferred embodiments illustrate adaptations of these compositions and methods. Nonetheless, from the description of these embodiments, other aspects of the invention can be made and/or practiced based on the description provided below.

### EXAMPLES

### Example 1

### Determination That CBZ Binds Avidin

In order to determine whether CBZ can bind to avidin, a solution of 10 mg/L CBZ dissolved in methanol was used in a HABA assay (Pierce Chemical Company, Pittsburgh, Pa.). When [2-(4′-hydroxyazobenzene) benzoic acid (HABA) is added in excess of avidin (Pierce Chemical Company, Pittsburgh, Pa.), an absorption at 500 nm is observed and a change in color occurs from yellow to red. This absorption is decreased proportionately when biotin is added since biotin displaces the HABA dye due to its higher affinity for avidin. The assay can be performed in a single cuvette by noting the A₅₀₀ of the avidin-HABA solution before and after addition of the biotin-containing sample; the change in absorbance can then be related to the amount of biotin in the sample by using the extinction coefficient of the complex.

The results of the assay showed that CBZ can also displace HABA, presumably because CBZ has an ureido group (the structural group of biotin that binds avidin) similar to biotin. The change in absorbance at 500 nm was 0.5633, indicating that CBZ does in fact bind avidin, and that it binds more tightly to avidin than does HABA, since it can apparently displace this molecule from avidin. This change in absorbance is equivalent to 0.17 μmoles biotin-competing capacity of CBZ/ml reaction mixture. This amount was present according to a calculation equation described by the manufacturer.

### Example 2

### Competitive Assay

Biotinylated bovine serum albumin (BSA) was synthesized by mixing 50 mL of 10 g/L BSA in ice-cold 0.1 mol/L NaHCO₃ (pH 7.5) with 5 mL of a 12 g/L N-hydroxysuccinimide ester (NHS-biotin) in dimethyl sulfoxide overnight at 4° C. The mixture was dialyzed for 48 h with gentle stirring at 4° C.

A flat bottom 96-well microtiter plate (Nunc Maxisorp, Fisher Scientific, Pittsburgh, Pa.) was coated with 200 μL of a 1:2 dilution of biotinylated BSA in coating buffer (50 mmol/L bicarbonate, pH 9.0) and incubated at 4° C. for at least 1 h (up to 48 h).

A 10 g/L stock of CBZ was made by dissolving CBZ in methanol and then this was diluted 1:2 in Hepes buffer (0.1 mol/L Hepes, 1 mol/L NaCl, pH 7.0) to make a concentration of 5 g/L. 5 μL of this stock was diluted in 295 μL serum (collected immediately after a rat had been killed by exsanguination and whole blood was allowed to clot 10 min before centrifugation at 10,000× g for 10 min, and serum recovered). This stock was then serially diluted in serum to have a standard curve with concentrations of 100-0.78 mg/L. This range is ideal since the therapeutic range for humans is 4-12 mg/L. The CBZ standard curve must be diluted in serum because the unknown samples to be tested will also be serum samples, which contain biotin. Since this assay is based on the binding of avidin to CBZ, the binding of CBZ to biotin in the sample must be accounted for (since the binding of avidin to biotin is much stronger). The aim in designing this assay was to make a standard curve in the same medium as the samples will be and then add enough avidin to bind all free biotin, with leftover avidin available for binding CBZ.

Once the standard curve was made, 100 μL of each standard (or unknown sample) was added into individual wells of a U-bottom 96-well microtiter plate (Pro-Bind, Becton Dickinson, Franklin Lakes, N.J.). Then 50 μL of a 1:20,000 dilution of Neutravidin-horseradish peroxidase (avidin-HRP, Pierce Chemical Company, Birmingham, Ala.) diluted in avidin buffer (0.1 mol/L Hepes, pH 7.0, 1 mol/L NaCl, 0.1% (w/v) BSA) was added to each well with standard or sample and incubated for 45 min-1 h at room temperature.

The coated flat bottom plate was washed three times with 0.05% (v/v) tween-20 and before the wells dried out, 100 μL of the contents of the U-bottom plate were added to the flat bottom plate and incubated for 45 min—up to 4 h. This flat bottom plate was washed with 0.05% (v/v) tween-20. 100 μL substrate solution (0.1 mol/L citric acid, 0.2 mol/L sodium phosphate, pH 5, 4.5 mmol/L o-phenylenediamine, 0.012% (v/v) H₂O₂) was then added to the plate and incubated for 45 min. After 45 min, 50 μL of 2 mol/L H₂SO₄ was added to the wells and the absorbance was measured at 490-650 nm in a plate reader spectrophotometer (Molecular Devices, Sunnyvale, Calif.).

### Example 3

### Standard Curve

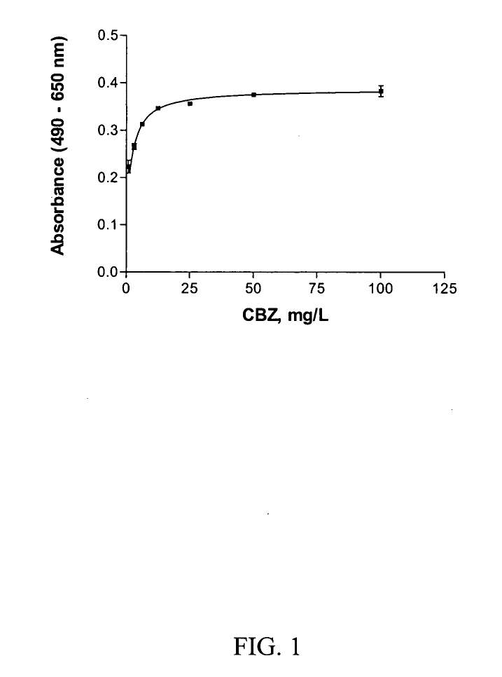

A standard curve was developed (as described in the methods above) with known concentrations of CBZ and several samples of serum CBZ from CBZ-treated rats were tested by this competitive assay and compared to values obtained by HPLC with UV detection. The results are shown in FIG. 1 and Table 1.

Testing samples of rat serum that had consumed 3.75 g CBZ/kg diet for 68 days and comparing CBZ concentration with value obtained from standard method of measuring CBZ using HPLC with UV detection as described previously (Rathman et al., J. Nutr. 132:3405-3410, 2002; Szabo and Browne, Clin. Chem. 28:100-104, 1982).

TABLE 1

| Sample | Competitive Assay | HPLC |
|---|---|---|
| Serum #1 | 4.8 mg/L | 4.7 mg/L |
| Serum #2 | 3.8 mg/L | 3.3 mg/L |
| Serum #3 | 3.0 mg/L | 3.3 mg/L |

### Example 4

### Benefits of the Assay

This assay might be used to measure other anticonvulsants with ureido groups similar to biotin, including primidone, phenytoin, progabide, phenobarbital, and oxcarbamazepine.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for detecting CBZ in a sample, the method comprising the steps of:
(A) providing a test sample;
(B) adding to the test sample an agent comprising avidin; and
(C) measuring the amount of CBZ specifically bound to avidin.

2. The method of claim 1, wherein the test sample comprises serum.

3. The method of claim 1, wherein the avidin is labeled with a detectable label.

4. The method of claim 1, wherein the avidin is selected from the group consisting of: avidin from an egg, streptavidin, and avidin derivatives.

5. The method of claim 1, wherein the method further comprises the steps of:
(D) providing at least one immobilized agent that binds avidin; and
(E) contacting the test sample and the agent comprising avidin to the at least one immobilized agent that binds avidin under conditions that allow binding of avidin to CBZ and binding of avidin to the at least one immobilized agent, wherein steps (D) and (E) are performed prior to the step (C) of measuring the amount of CBZ specifically bound to avidin.

6. The method of claim 5, wherein the step (C) of measuring the amount of CBZ specifically bound to avidin comprises measuring the amount of avidin bound to the at least one immobilized agent.

7. The method of claim 6, wherein the at least one immobilized agent is biotin.

8. The method of claim 7, wherein the biotin is conjugated to bovine serum albumin.

9. The method of claim 8, wherein the combination of biotin and bovine serum albumin is contacted with a plastic dish.

10. The method of claim 6, wherein the test sample comprises serum.

11. The method of claim 6, wherein the avidin is labeled with a detectable label.

12. The method of claim 11, wherein the detectable label is selected from the group consisting of: a radioisotope, a fluorescent compound, a bioluminescent compound, a chemiluminescent compound, colloidal gold, a magnetic particle, and an enzyme.

13. The method of claim 12, wherein the detectable label comprises horseradish peroxidase.

14. The method of claim 5, wherein the step (E) of contacting the test sample and the agent comprising avidin to the at least one immobilized agent that binds avidin under conditions that allow binding of avidin to CBZ and binding of avidin to the at least one immobilized agent comprises mixing the test sample and the avidin before contacting the test sample and the avidin to the at least one immobilized agent that binds avidin.

15. The method of claim 11, wherein measuring the amount of avidin bound to the at least one immobilized agent comprises contacting the avidin bound to the at least one immobilized agent with a substrate molecule that interacts with the detectable label yielding a detectable signal.

16. The method of claim 15, wherein the detectable signal is selected from the group consisting of: light, color and radioactivity.

17. The method of claim 16, wherein the detectable signal is quantified using a spectrophotometer.

18. The method of claim 15, wherein the level of signal is proportional to the level of CBZ in the test sample.

19. The method of claim 1, wherein steps (A), (B), and (C) are performed in a competitive binding assay.

20. The method of claim 19, wherein the competitive binding assay is selected from the group consisting of: radioimmunoassay, radioligand binding assay, fluorescence polarization binding assay, ELISA, microplate reader-based assay, fluorimetric displacement assay, FRET assay, affinity chromatography-based assay, non-chromatographic affinity assay, protein microarray assay, and fluorimmunoassay.

21. The method of claim 1, wherein steps (A), (B), and (C) are performed in a non-competitive binding assay.

22. The method of claim 21, wherein the non-competitive binding assay is selected from the group consisting of: agglutination assay, enzyme immunoassay, immunometric assay, radio immunoassay, ELISA, fluorescent immunoassay, chemiluminescent assay, calorimetric assay, plasmon resonance assay, FRET assay, lateral flow assay, and flow cytometry assay.

23. A kit for detecting CBZ in a sample comprising:
(A) a plurality of immobilized agents that bind avidin;
(B) a plurality of label-conjugated avidins;
(C) a wash solution;
(D) a reagent for detection of the complexes formed between the label-conjugated avidins and the plurality of immobilized agents that bind avidin; and
(E) instructions for use.

24. The kit of claim 23, wherein the plurality of immobilized agents that bind avidin are conjugated to a solid support.

25. The kit of claim 24, wherein the solid support comprises a plastic dish.
